(19) United States
(12) Patent Application Publication
(10) Pub. No.: US 2024/0401124 A1
(43) Pub. Date: Dec. 5, 2024

(54) NUCLEAR DNA-ANTIBODY SEQUENCING FOR JOINT PROFILING OF GENOTYPE AND PROTEIN IN SINGLE NUCLEI

(71) Applicants: CZ BIOHUB SF , LLC, San Francisco, CA (US); THE REGENTS OF THE UNIVERSITY OF CALIFORNIA, Oakland, CA (US)

(72) Inventors: Adam Abate, Oakland, CA (US); Harish Vasudevan, Oakland, CA (US); Benjamin Demaree, Oakland, CA (US); Cyrille L. Delley, Oakland, CA (US)

(21) Appl. No.: 18/698,898

(22) PCT Filed: Oct. 18, 2022
(86) PCT No.: PCT/US2022/047005

§ 371 Date: Apr. 5, 2024

Related U.S. Application Data
(60) Provisional application No. 63/257,426, filed on Oct. 19, 2021.

(51) Int. Cl. C12Q 1/6869; C12Q 1/6806; G01N 33/68

(52) U.S. Cl. CPC C12Q 1/6869; C12Q 1/6806; G01N 33/6854

(57) ABSTRACT

The present disclosure provides materials and methods for contemporaneously analyzing genotype and phenotype from frozen specimens, the present disclosure can be extended to any biologic context where obtaining nucleic acid and protein information from single nuclei is valuable. As used herein, the method is termed nuclear DAb-seq (nDAb-seq).

### CROSS-REFERENCE TO RELATED APPLICATIONS

This application claims the benefit of U.S. Provisional Application No. 63/257,426, filed Oct. 19, 2021, the entire contents of which are fully incorporated herein by reference

### FIELD

The present disclosure relates generally to methods for joint (e.g., simultaneously or contemporaneously) nucleic acid-protein profiling from single nuclei, which can be applied to all manner of biological samples (e.g. frozen tumor specimens, infections disease samples).

### BACKGROUND

DNA mutations leading to aberrant protein expression underlie tumorigenesis and cancer progression, yet conventional bulk sequencing and immunohistochemistry techniques fails to capture the full complexity of a given tumor. DNA-Antibody sequencing (DAb-seq; Demaree, B., et al., Nature Communications. 2021. PMID: 33707421) enables the determination of genotype and phenotype from the same cell. However, current technical limitations of the DAb-seq workflow require fresh samples with intact cells, limiting analysis of frozen tumor specimens in retrospective tissue banks.

### SUMMARY OF THE INVENTION

One embodiment of the present disclosure provides a method of determining (i) the presence of at least one biomolecule and (ii) the sequence of at least one nucleic acid from a single nuclei, said method comprising the steps of: (a) preparing single nuclei from a sample comprising multiple cells; (b) contacting the nuclei with at least one affinity reagent comprising a first barcode oligonucleotide and capable of binding to the at least one biomolecule, wherein said contacting occurs under conditions that allow binding of the at least one affinity reagent to the at least one biomolecule; (c) sequencing at least one nucleic acid from the nuclei under conditions that allow determining the presence of at least one biomolecule and the sequence of at least one nucleic acid from a single nuclei.

In another embodiment, the presence of the at least one biomolecule and the sequence of the at least one nucleic acid from a single nuclei is determined by the combination of (i) detection of the barcoded affinity reagent binding to the biomolecule and (ii) a sequencing reaction comprising a technique selected from the group consisting of scRNA-seq, scDNA-seq, Ab-seq, ATAC-seq, cut and run sequencing, and cut and tag sequencing.

In still another embodiment, the preparing single nuclei in step (a) comprises permeabilizing cells and optionally fixing nuclei from said cells. In another embodiment, an aforementioned method is provided wherein the sample is a tissue sample. In one embodiment, the sample is a cell line. In another embodiment, the tissue sample is a frozen tissue sample. In still another embodiment, the tissue sample is a human or animal biopsy. In yet another embodiment, the tissue sample is a solid tumor sample. The present disclosure further provides a method wherein the biopsy comprises a sample from a tumor, lymphatic tissue, infected tissue, immune infiltrated tissue, central nervous system tissue, digestive system tissue, developing tissue; whole animal section, and microbial community. In another embodiment, the tissue sample is subjected to freezing and/or slicing. In still another embodiment, the tissue sample, prior to preparing step (a), is subjected to a treatment selected from the group consisting of imaging, spectroscopy, mass spectroscopy, enzymatic assays and FISH.

In various embodiments, the present disclosure provides an aforementioned method wherein the biomolecule is selected from the group consisting of a nucleic acid, a lipid, a small molecule, a sugar, and a protein. In one embodiment, the biomolecule is a protein. In another embodiment, the protein is a nuclear protein, a transcription factor, a signal transduction protein, an epigenetic regulator protein, and/or a histone modification protein.

In some embodiments, an aforementioned method is provided wherein the sequencing comprises a technique selected from the group consisting of scRNA-seq, scDNA-seq, Ab-seq, ATAC-seq, cut and run sequencing and combinations thereof. In other embodiments, an aforementioned method is provided wherein the nucleic acid is selected from the group consisting of DNA, RNA, and mRNA.

The present disclosure also provides, in various embodiments, an aforementioned method wherein the presence of 2, 3, 4, 5, 6, 7, 8, 9, 10 or more biomolecules are determined. In other embodiments, the presence and/or sequence of 2, 3, 4, 5, 6, 7, 8, 9, 10 or more nucleic acids are determined.

In other embodiments, an aforementioned method is provided wherein the preparing in step (a) comprises one or more of (1) mechanically mincing and/or cutting a frozen tumor sample; and/or (2) permeabilizing cells in the sample under conditions that allow isolation of intact nuclei from said cells; and/or (3) fixing nuclei. In one embodiment, the permeabilizing comprises contacting the cells with a buffer comprising one or more of Tween-20, dithiothreitol (DTT), bovine serum albumin (BSA), hydroxyethyl piperazineethanesulfonic acid (HEPES), sodium chloride, calcium chloride, magnesium chloride, and water. In still another embodiment, the fixing comprises contacting the cells with a buffer comprising one or more of DSP, DMSO, methanol, paraformaldehyde (PFA), ethanol, acetic acid and trehalose.

The present disclosure also provides, in various embodiments, an aforementioned method wherein the sequencing of step (c) comprises one or more of: (1) encapsulating one or more nuclei into droplets in a first emulsion wherein said droplets contain beads comprising barcoded oligonucleotides; and/or (2) releasing the barcoded oligonucleotides of (1) from said beads under conditions that allow the oligonucleotide to hybridize to a corresponding sequence associated with the one or more nuclei; and/or (3) collecting the nuclei from the emulsion of (1) under conditions that allow the barcoded oligonucleotides to fuse with the corresponding oligonucleotide in the nuclei; and/or (4) re-encapsulating in a second emulsion the nuclei into droplets and wherein said droplets contain beads comprising barcoded oligonucleotides; and/or (5) processing and collecting the re-encapsulated nuclei under conditions that allow barcoding of amplified genomic content; and/or (6) sequencing the genomic content of (5).

### BRIEF DESCRIPTION OF THE DRAWINGS

FIG. 1 shows an exemplary workflow to use isolated nuclei in high-throughput genomics and resulting in simultaneous measurement of DNA and protein from single cells.

FIG. 2 shows a successful nuclei isolation and nuclear DNA Antibody sequencing (nDAb-seq) workflow permitting simultaneous measurement of both DNA (top data panels) and protein (bottom data panels) from single nuclei.

FIGS. 3A and 3B show (FIG. 3A) a representative single nuclear dissociation demonstrating DAPI stained nuclei (in blue) super imposed on light field image of the sample, (FIG. 3B) a lower magnification image of intact DAPI stained nuclei (in blue) demonstrating sufficient yield from a tumor specimen.

### DETAILED DESCRIPTION

The present disclosure addresses the aforementioned shortcomings by providing compositions and methods to contemporaneously analyze genotype and phenotype from frozen specimens. In various embodiments, and as will be appreciated by those in the field, the present disclosure can be extended to any biologic context where obtaining DNA and protein information from single nuclei is valuable. As used herein, the method is termed nuclear DAb-seq (nDAb-seq).

Frozen clinical specimens are critical for molecular profiling yet recovery of intact cells from such samples is limited. Therefore, approaches that permit profiling of DNA genotype and protein abundance from single nuclei, rather than single cells, are critical. The current methods that are used to analyze frozen specimens analyze genotype and protein expression primarily on the bulk level, and while single cell genotype/protein technologies do not permit analysis of single nuclei, for example from frozen tumor specimens. nDAb-seq extends single DNA/phenotype profiling to single nuclei. Potential applications include tumor profiling and infectious disease diagnosis/prognosis are contemplated herein.

As described herein, following the preparation of single nuclei, and prior to the sequencing methods, there are multiple techniques and methods that can be used to derive a mode of multimodal analysis (i.e., in addition to the sequencing). Permeabilization and fixation, along with identifying the optimal antibody concertation during the staining and blocking steps described herein, were technical hurdles and provided herein are appropriate buffers and conditions. The antibody, if used according to various embodiments, is used as a label and not necessarily to address or identify the presence of an expressed protein, unlike prior techniques described in the art. Whereas such prior methods focused on proteins on intact, live cells, the present disclosure provides methods for using nuclei and affinity reagents to obtain genotype and phenotype information.

The present disclosure provides, in various embodiments described herein, methods to meet the aforementioned need in the art by performing, for example, joint (e.g., simultaneously or contemporaneously) DNA-protein profiling from single nuclei. By way of example, in one embodiment, the present disclosure provides a method of determining (i) the presence of at least one biomolecule and (ii) the sequence of at least one nucleic acid from a single nuclei, said method comprising the steps of: (a) preparing single nuclei from a sample comprising multiple cells; (b) contacting the nuclei with at least one affinity reagent comprising a first barcode oligonucleotide and capable of binding to the at least one biomolecule, wherein said contacting occurs under conditions that allow binding of the at least one affinity reagent to the at least one biomolecule; (c) sequencing at least one nucleic acid from the nuclei under conditions that allow determining the presence of at least one biomolecule and the sequence of at least one nucleic acid from a single nuclei.

As used herein, an affinity reagent can be an antibody, aptamer, label, or other reagent capable of binding to one or more biomolecules including nucleic acids and proteins. In one embodiment the affinity reagent is an antibody. In another embodiment, the affinity reagent can be the natural “stickiness’ of the nucleic acid (e.g., DNA) itself (i.e., DNA's propensity to “stick” to the nucleus). In another embodiment, the affinity reagent is an aptamer. In various embodiment, any aforementioned affinity reagent further includes or otherwise comprises a barcode or barcoding reagent as described herein and known in the art.

Sequencing reactions and techniques are well known in the art and include, without limitation, scRNA-seq, scDNA-seq, Ab-seq, ATAC-seq, cut and run sequencing, and cut and tag sequencing.

As used herein, scRNA-seq is a method that permits measurement of RNA molecules from each individual cell within a multicellular sample, providing whole transcriptome from each cell within a tissue An exemplary method is disclosed in Macosko, E. et al., Cell, 2015, 161(5):1202-1214, which is incorporated by reference herein.

As used herein, scDNA-seq is a method that permits measurement of DNA molecules from each individual cell within a multicellular sample, providing mutational information from each cell within a tissue. An exemplary method is disclosed in Pellegrino, M., et al., Genome Res., 2018, 28(9):1345-1352, which is incorporated by reference herein.

As used herein, Ab-seq is a method that permits measurement of protein molecules from each individual cell within a multicellular sample, providing protein abundance estimates from each cell within a tissue. An exemplary method is disclosed in Shahi, P., et al., Sci Rep., 7:44447, which is incorporated by reference herein.

As used herein, ATAC-seq is a method that permits measurement of chromatin accessibility as a proxy for active gene expression and transcriptional regulation from a tissue. An exemplary method is disclosed in Cusanovich, D., et al., Cell, 174(5):1309-1324, which is incorporated by reference herein.

As used herein, cut and run sequencing is a method that permits measurement of interactions between proteins and DNA within a tissue to determine regulatory interactions that affect gene expression. An exemplary method is disclosed in Skene, P., et al., Nat. Protocol., 13(5):1006-1019, which is incorporated by reference herein.

As used herein, cut and tag sequencing is a method permits measurement of interactions between proteins and DNA within a tissue to determine regulatory interactions that affect gene expression which differs from cut and run by leveraging a different enzyme and tagmentation process to facilitate single cell measurements. An exemplary method is disclosed in Kaya-Okur, H., et al., Nat. Commun., 10(1):1930, which is incorporated by reference herein.

As used herein, the term “sample” or “biological sample” or “tissue sample” encompasses a variety of sample types obtained from a variety of sources, which sample types contain biological material. For example, the term includes biological samples obtained from a mammalian subject, e.g., a human subject, and biological samples obtained from a food, water, or other environmental source, etc. The definition encompasses blood and other liquid samples of biological origin, as well as solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “sample” or “biological sample” encompasses a clinical sample, and also includes cells in culture, cell supernatants, cell lysates, cells, serum, plasma, biological fluid, and tissue samples. “Sample” and “biological sample” includes cells, e.g., bacterial cells or eukaryotic cells; biological fluids such as blood, cerebrospinal fluid, semen, saliva, and the like; bile; bone marrow; skin (e.g., skin biopsy); and viruses or viral particles obtained from an individual. In one embodiment the tissue sample is a frozen tumor tissue sample. In another embodiment, the sample can be comprised of a cell line that is, for example, grown under tissue culture conditions.

As described more fully herein, in various aspects the subject methods may be used to detect a variety of components or “biomolecules” from such biological samples. Biomolecules of interest include, but are not necessarily limited to, polynucleotides (e.g., DNA and/or RNA), polypeptides (e.g., peptides and/or proteins), and many other components that may be present in the biological sample.

The terms “polynucleotide” and “nucleic acid” and “target nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 10⁹ nucleotides or larger. Polynucleotides and nucleic acids include RNA, cDNA, genomic DNA. In particular, the polynucleotides and nucleic acids, is used herein to refer to a binding moiety used in the methods described herein and/or as a target of the methods described herein (e.g., a target whose location and sequence is determined by practicing the methods described herein). In some embodiments, the nucleic acid is rRNA, tRNA, mRNA, or mtRNA.

The term “oligonucleotide” refers to a polynucleotide of from about six (6) to about one hundred (100) nucleotides or more in length. Thus, oligonucleotides are a subset of polynucleotides. Oligonucleotides can be synthesized manually, or on an automated oligonucleotide synthesizer (for example, those manufactured by Applied BioSystems (Foster City, CA)) according to specifications provided by the manufacturer or they can be the result of restriction enzyme digestion and fractionation.

The term “protein” or “protein of interest” (e.g., as it relates to a (target) biomolecule) refers to a polymer of amino acid residues, wherein a protein may be a single molecule or may be a multi-molecular complex. The term, as used herein, can refer to a subunit in a multi-molecular complex, polypeptides, peptides, oligopeptides, of any size, structure, or function. It is generally understood that a peptide can be 2 to 100 amino acids in length, whereas a polypeptide can be more than 100 amino acids in length. A protein may also be a fragment of a naturally occurring protein or peptide. The term protein may also apply to amino acid polymers in which one or more amino acid residues is an artificial chemical analogue of a corresponding naturally occurring amino acid. A protein can be wild-type, recombinant, naturally occurring, or synthetic and may constitute all or part of a naturally-occurring, or non-naturally occurring polypeptide. The subunits and the protein of the protein complex can be the same or different. A protein can also be functional or non-functional.

Generally, other nomenclature used herein and many of the laboratory procedures in cell culture, molecular genetics and nucleic acid chemistry and hybridization, which are described below, are those well-known and commonly employed in the art. (See generally Ausubel et al. (1996) supra; Sambrook et al, Molecular Cloning: A Laboratory Manual, Second Edition, Cold Spring Harbor Laboratory Press, New York (1989), which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

“Detecting” or “determining” or “measuring” as used herein generally means identifying the presence of a target, such as a target nucleic acid or protein or biomolecule. In various embodiments, detection signals are produced by the methods described herein, and such detection signals may be optical signals which may include but are not limited to, colorimetric changes, fluorescence, turbidity, and luminescence. Detecting, in still other embodiments, also means quantifying a detection signal, and the quantifiable signal may include, but is not limited to, transcript number, amplicon number, protein number, and number of metabolic molecules. In this way, sequencing or bioanalyzers are employed in certain embodiments.

According to some embodiments of the present disclosure, a lysing reagent is used in the methods. Lysing agents may include, for example chemical lysis, such as SDS, detergents, alkaline, and acid; biological lysis, such as lysis enzymes, viruses, and phages; and physical lysis such as beads beating, grinding, frozen-thaw, and sonication, heating, cutting, and laser or ion beams.

According to some embodiments of the present disclosure, single nuclei are isolated from a sample, such as a frozen tumor sample. An exemplary workflow is as follows:
Mechanically mince and/or cut frozen specimen (e.g., tumor sample or other tissue sample)
Permeabilize with TSH buffer (Tween-20+SH buffer) with appropriate salts as described herein and known in the art
Fix with DSP in DMSO and appropriate salts as described herein and known in the art, followed by methanol. The combination of DSP followed by methanol is critical depending on the sample. The DMSO reagent can be optional but is important for certain downstream sequencing techniques and other analytical techniques such as ATAC-seq and CUT&Run versions.
Stain with antibody panel against nuclear proteins
Nuclei now ready for additional downstream measurements, including, but not limited to, scRNA-seqs, scDNA-seq, Ab-seq, ATAC-seq, Cut and run sequencing, and combinations thereof.

According to other embodiments of the present disclosure, high-throughput genomics, such as workflows described herein and below, is used to extract nuclei and determine sequence and protein expression metrics from single cells:
Extract nuclei as described herein and above and fix with any method or optionally use unfixed
Load a plurality, e.g. 1-20 nuclei on average, into droplets together with a plurality, e.g., 1-20 (small) barcode beads (hash beads), where bead is each charged with unique barcode sequences
Release the barcodes (hashes) from the bead under conditions which allow attachment to the genomic content
Break the emulsion, collect the nuclei and either perform bulk manipulation (such as reverse transcription of the RNA, tagmentation of the DNA) before re-encapsulation in a second dropmaker, or alternatively directly re-encapsulate. Again the nuclei are loaded at preferentially ˜1-20 nuclei per droplet and combined with one (or several) barcode beads
In the second (e.g., re-encapsulate) droplet, the barcode(s) are released from the bead(s) and used in a PCR or reverse transcription reaction to label the genomic content of interest (DNA, RNA, antibody-tags, etc.) together with the hashes from the first droplet.
Now the droplet contains a genomic library in which each molecule forms a physical unit of barcode+hash+genomic read. This library is collected, prepared for sequencing, and sequenced. Regaining single cell identity from this process is trivial if in both steps a single hash bead/barcoding bead is used. In this way, molecules from single cells are fully defined by the unique hash+barcode combination.
If in either step multiple beads are used, reads from single cells are “smeared out” over multiple hash+barcode combinations. However, provided that each hash or barcode was used only once, this ambiguity can be collapsed considering that read distributions from the same cell are highly correlated. This workflow increases the throughput of droplet microfluidic workflows by ˜10-1,000 fold without loss of single cell resolution.

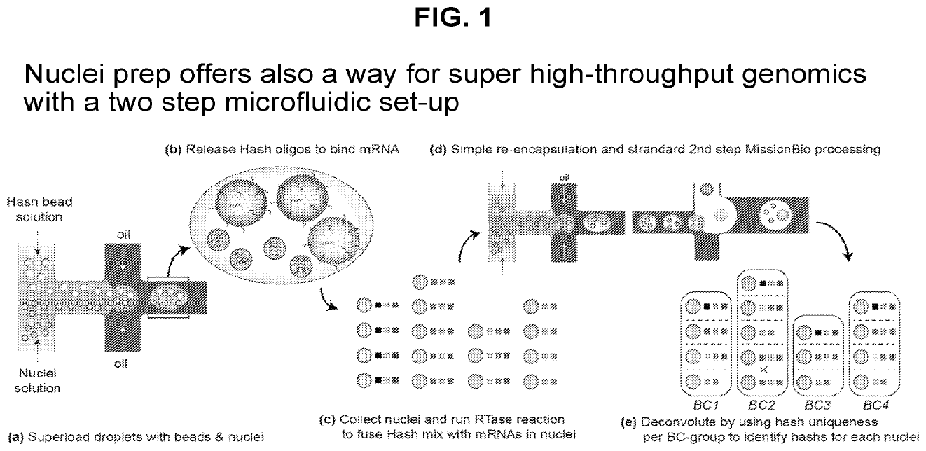

In some embodiments, an exemplary workflow to use isolated nuclei in high-throughput genomics resulting in simultaneous measurement of DNA and protein from single cells is provided in FIG. 1.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

### EXAMPLES

### Example 1

### Nuclei Preparation and Antibody Staining

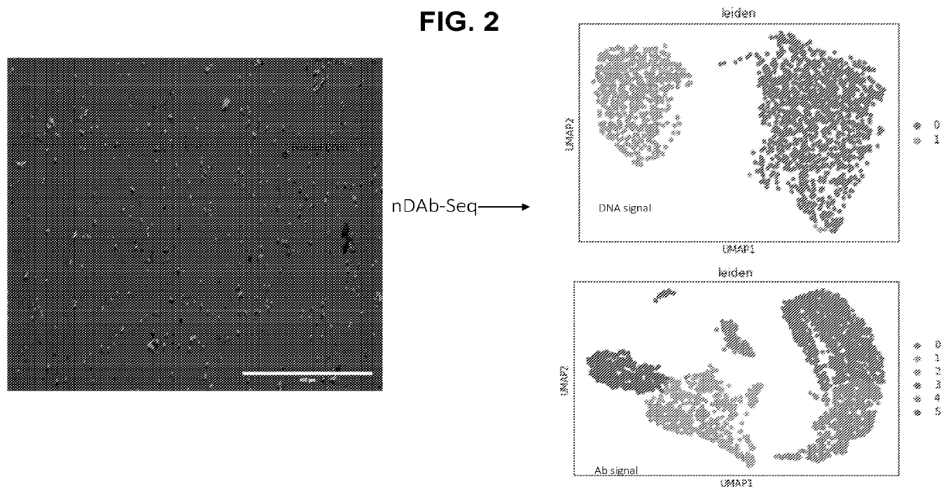

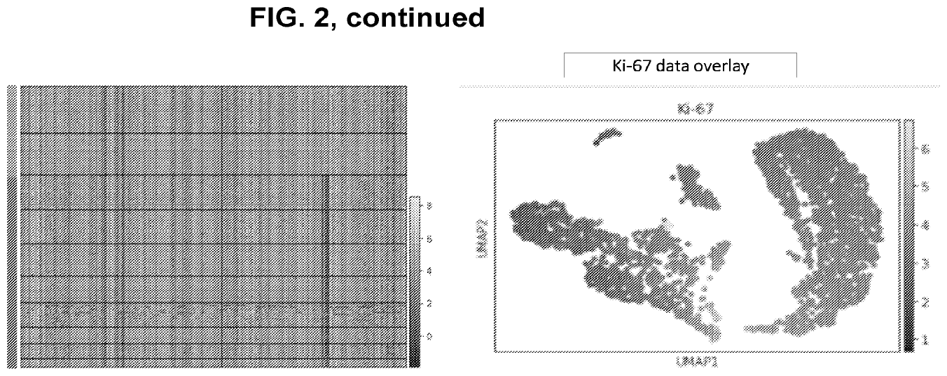

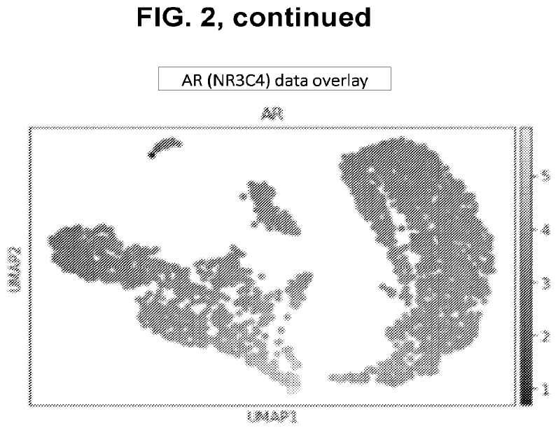

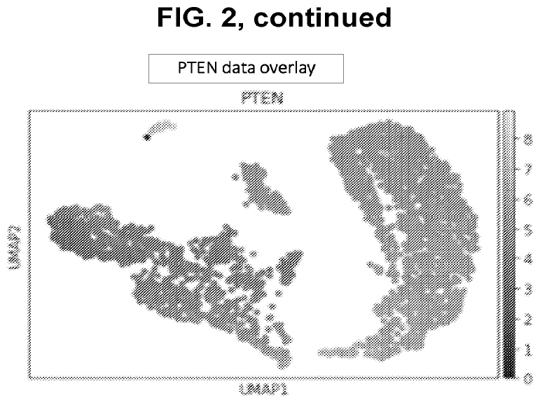

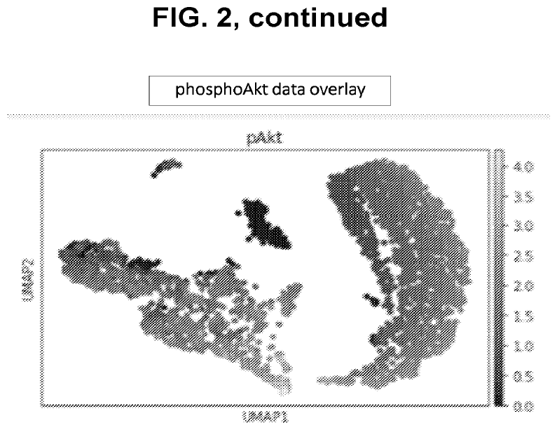

This Example provides the material (compositions, buffers, and the like) and methods including a protocol for the isolation of nuclei, and subsequent staining. FIG. 2 shows a successful nuclei isolation and nuclear DNA Antibody sequencing (nDAb-seq) workflow permitting simultaneous measurement of both DNA (top data panels) and protein (bottom data panels) from single nuclei.

### Sample and Nuclei Prep Protocol:

1. Digest frozen in 1 ml TSH nuclei extraction buffer and cut for 10 min on ice in 2 ml protein lo bind tube.

| | stock | 2x | 1x | units | volume | |
|---|---|---|---|---|---|---|
| 1 x TSH buffer | | | | | | |
| NaCl | 1000 | 292 | 146 | mM | 292 | ul |
| Hepes pH 7.3 | 1000 | 20 | 10 | mM | 20 | ul |
| CaCl2 | 500 | 2 | 1 | mM | 4 | ul |
| MgCl2 | 1000 | 42 | 21 | mM | 42 | ul |
| 2x SH buffer | 2 | — | 1 | x | 1000 | ul |
| Tween-20 | 10 | 0.06 | 0.03 | % | 6 | ul |
| BSA | 5 | 0.02 | 0.01 | % | 4 | ul |
| DTT | 1000 | 1 | 0.5 | mM | 1 | ul |
| RNasin plus | 40 | 0.08 | 0.04 | U/ul | 2 | ul |
| H2O | — | — | — | — | 987 | ul |
| tot | | | | | 2000 | ul |
| 2x SH buffer | | | | | | |
| NaCl | 2000 | 292 | 146 | mM | 1460 | ul |
| Hepes pH 7.3 | 1000 | 20 | 10 | mM | 200 | ul |
| CaCl2 | 2000 | 2 | 1 | mM | 10 | ul |
| MgCl2 | 1000 | 42 | 21 | mM | 420 | ul |
| H2O | — | — | — | — | 7910 | ul |
| tot | | | | | 10000 | ul |

2. Filter through a 50 um filter
3. Wash with 1 ml TSH and 3 ml 1×SH into 5 mL protein Lo Bind tube
4. Pelleted 5 min 500 g and resuspended in 0.75 ml by gently pipetting SH
5. Added 2×PFA solution, kept on ice and pipetted gently for 1:30 min
6. Added 1 ml stop mix and pelleted as before.

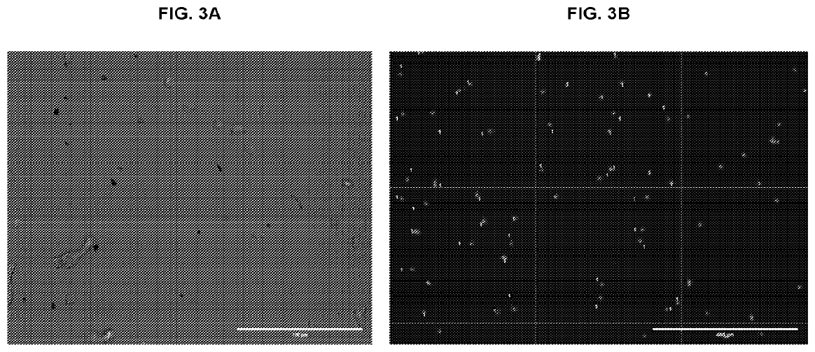

As shown in FIG. 3A, the above approach permits robust single nuclei dissociation (stained with DAPI shown in blue) while maintaining nuclear integrity.

| 2x PFA fix | | | | | | |
|---|---|---|---|---|---|---|
| 2x SH buffer | 2 | 1 | 0.5 | x | 375 | ul |
| PFA | 16 | 0.2 | 0.1 | % | 9.375 | ul |
| Acetic Acid | 100 | 0.15 | 0.075 | % | 1.125 | ul |
| H2O | — | — | — | — | 364.5 | ul |
| tot | | | | | 750 | ul |

| 2x stop mix | | | | | | |
|---|---|---|---|---|---|---|
| BSA | 5 | 0.4 | 0.2 | mg/ml | 80 | ul |
| 2x ST buffer | 2 | 1 | 0.1 | % | 500 | ul |
| H2O | — | — | — | — | 420 | ul |
| tot | | | | | 1000 | ul |

Resuspended in 2 ml KINfix (due to the encouraging immuno fluorescence achieved on my FACS and in the original publication, EtOH is fixative of choice for Ki67 according to BD and older publications suggesting that AcCOOH is necessary in alcohol fixatives to kill residual RNAse activity and helps precipitate nucleic acids) and incubated 30 min on ice.

| 2x KINfix | stock | 1.1x | 1x | | | |
|---|---|---|---|---|---|---|
| EtOH | 100 | 68.75 | 62.5 | % | 1375 | ul |
| Acetic acid | 100 | 7.37 | 6.7 | % | 147.4 | ul |
| Trehalose | 1.6 | 0.1925 | 0.175 | M | 240.625 | ul |
| Water | — | | — | | 236.975 | ul |
| tot | | | | | 2000 | ul |

### Staining and Antibody Preparation Protocol:

While on ice prepared antibodies:
Nuclear panel—4 ul of each: Ki67, Suz12, pTEN, RB, Erg, p53, P16, AR, Cdk1b, Myc, FoxA, p63, Nkx3.1n IgG1 (didn't find H3Kme3)->44 ul total.
Cytosol+lymphocytes—2 ul each, 4 ul of CD4 and CD8: pAKT, pERK1/2, pp38; PD-L1, PD-1, CTLA4, CD5, CD4, CD8, CD3, CD7 (CD56 clashes with some barcode).->26 ul

### Combined 4× Recipe for Nuclear Panel, or 2× for Cyto&Lympho Panel:

| nuclear | Cyto&Lympho | |
|---|---|---|
| 44 ul | 26 ul | Ab-pool (~0.1 mg/ml) (in PBS) |
| 4 ul | 2 ul | ATP (10 mM −>0.5 mM) |
| 4 ul | 2 ul | CITE-seq splint (100 uM −>2.5 uM) |
| 4 ul | 2 ul | CITE-seq adapter (100 uM −>2.5 uM) |
| 8 ul | 4 ul | MgCl2 (50 uM −>5 mM) |
| 2 ul | 1 ul | salt T4 ligase |

Incubated 30 min at RT, afterwards kept on ice (after ˜4 h when experiment was done, added 10 mM (final) EDTA to quench ligation).

Pelleted at 500 g, 5 min, 4 C removed supernatant, added 1 ml wash solution without resuspending cell pellet to dilute EtOH (not sure if that produced some dumping, should have had RNAse inhibitor and 3 mM MgCl2 in the buffer). Pelleted again and resuspended in 400 ul blocking solution. Incubated 10 min on ice.

| 1x Block/stain solution | | | | | | |
|---|---|---|---|---|---|---|
| BSA | 5 | 2 | 1 | mg/ml | 100 | ul |
| SSDNA | 25 | 2 | 1 | x | 20 | ul |
| RNasin plus | 40 | 0.08 | 0.04 | U/ul | 0.5 | ul |
| FcX | 100 | 2 | 1 | x | 5 | ul |
| Myeloid block | 100 | 2 | 1 | x | 5 | ul |
| MgCl2 | 1000 | 6 | 3 | mM | 1.5 | ul |
| PBS | — | — | — | — | 369.5 | ul |
| tot | | | | | 500 | ul |

| 1x wash solution | | | | | | |
|---|---|---|---|---|---|---|
| BSA | 5 | 0.4 | 0.2 | mg/ml | 120 | ul |
| RNasin plus | 40 | 0.08 | 0.04 | U/ul | 3 | ul |
| PBS | — | — | — | — | 2877 | ul |
| tot | | | | | 3000 | ul |

After 10 min added 16 ul prepared nuclear panel, 4 ul of old nuclear panel (because it contains H3Kme3), and 12 ul Cyto&Lympho panel (in case some lymphocytes made it through intact). Incubated 1 h on ice, pelleted and washed 2× with 1 ml wash buffer. Re-suspended in 100 ul and wash buffer and took count at 1500 c/ul. As shown in FIG. 3B, the re-suspended nuclei maintain integrity (as stained by DAPI shown in blue) and are of sufficient quantity and quality to permit downstream analysis as depicted by the workflow in FIG. 1.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method of determining (i) the presence of at least one biomolecule and (ii) the sequence of at least one nucleic acid from a single nuclei, said method comprising the steps of:
(a) preparing single nuclei from a sample comprising multiple cells;
(b) contacting the nuclei with at least one affinity reagent comprising a first barcode oligonucleotide and capable of binding to the at least one biomolecule, wherein said contacting occurs under conditions that allow binding of the at least one affinity reagent to the at least one biomolecule;
(c) sequencing at least one nucleic acid from the nuclei under conditions that allow determining the presence of at least one biomolecule and the sequence of at least one nucleic acid from a single nuclei.

2. The method of claim 1, wherein said the presence of the at least one biomolecule and the sequence of the at least one nucleic acid from a single nuclei is determined by the combination of (i) detection of the barcoded affinity reagent binding to the biomolecule and (ii) a sequencing reaction comprising a technique selected from the group consisting of scRNA-seq, scDNA-seq, Ab-seq, ATAC-seq, cut and run sequencing, and cut and tag sequencing.

3. The method of claim 1 or 2, wherein said preparing single nuclei in step (a) comprises permeabilizing cells and optionally fixing nuclei from said cells.

4. The method of any one of claims 1-3, wherein the sample is a tissue sample.

5. The method of claim 4, wherein the sample is a cell line.

6. The method of claim 4, wherein the tissue sample is a frozen tissue sample.

7. The method of claim 4 or 6, wherein the tissue sample is a human or animal biopsy.

8. The method of claim 7, wherein the tissue sample is a solid tumor sample.

9. The method of claim 7, wherein the biopsy comprises a sample from a tumor, lymphatic tissue, infected tissue, immune infiltrated tissue, central nervous system tissue, digestive system tissue, developing tissue; whole animal section, and microbial community.

10. The method of claim 4, wherein the tissue sample is subjected to freezing and/or slicing.

11. The method of claim 4, wherein the tissue sample, prior to preparing step (a), is subjected to a treatment selected from the group consisting of imaging, spectroscopy, mass spectroscopy, enzymatic assays and FISH.

12. The method of any one of claims 1-11, wherein the biomolecule is selected from the group consisting of a nucleic acid, a lipid, a small molecule, a sugar, and a protein.

13. The method of claim 12, wherein the biomolecule is a protein.

14. The method of claim 13, wherein the protein is a nuclear protein, a transcription factor, a signal transduction protein, an epigenetic regulator protein, and/or a histone modification protein.

15. The method of any one of claims 1-14, wherein the sequencing comprises a technique selected from the group consisting of scRNA-seq, scDNA-seq, Ab-seq, ATAC-seq, cut and run sequencing and combinations thereof.

16. The method of any one of claims 1-15, wherein the nucleic acid is selected from the group consisting of DNA, RNA, and mRNA.

17. The method of any one of claims 1-16, wherein the presence of 2, 3, 4, 5, 6, 7, 8, 9, 10 or more biomolecules are determined.

18. The method of any one of claims 1-17, wherein the presence and/or sequence of 2, 3, 4, 5, 6, 7, 8, 9, 10 or more nucleic acids are determined.

19. The method of any one of claims 1-18, wherein the preparing in step (a) comprises one or more of (1) mechanically mincing and/or cutting a frozen tumor sample; and/or (2) permeabilizing cells in the sample under conditions that allow isolation of intact nuclei from said cells; and/or (3) fixing nuclei.

20. The method of claim 19, wherein the permeabilizing comprises contacting the cells with a buffer comprising one or more of Tween-20, dithiothreitol (DTT), bovine serum albumin (BSA), hydroxyethyl piperazineethanesulfonic acid (HEPES), sodium chloride, calcium chloride, magnesium chloride, and water.

21. The method of any one of claims 19-20, wherein the fixing comprises contacting the cells with a buffer comprising one or more of DSP, DMSO, methanol, paraformaldehyde (PFA), ethanol, acetic acid and trehalose.

22. The method of any one of claims 1-21, wherein the sequencing of step (c) comprises one or more of:
(1) encapsulating one or more nuclei into droplets in a first emulsion wherein said droplets contain beads comprising barcoded oligonucleotides; and/or
(2) releasing the barcoded oligonucleotides of (1) from said beads under conditions that allow the oligonucleotide to hybridize to a corresponding sequence associated with the one or more nuclei; and/or
(3) collecting the nuclei from the emulsion of (1) under conditions that allow the barcoded oligonucleotides to fuse with the corresponding oligonucleotide in the nuclei; and/or
(4) re-encapsulating in a second emulsion the nuclei into droplets and wherein said droplets contain beads comprising barcoded oligonucleotides; and/or
(5) processing and collecting the re-encapsulated nuclei under conditions that allow barcoding of amplified genomic content; and/or
(6) sequencing the genomic content of (5).
